(19) United States
(12) Patent Application Publication
(10) Pub. No.: US 2005/0220782 A1
(43) Pub. Date: Oct. 6, 2005

(54) Method of treatment for stroke employing administration of defibrinogenating agents to achieve a specific defibrinogenation pattern

(76) Inventors: Stephen J. Petti, East Durham, NY (US); Samn Raffaniello, East Durham, NY (US)

Correspondence Address: HESLIN ROTHENBERG FARLEY & MESITI PC, 5 COLUMBIA CIRCLE, ALBANY, NY, 12203, US

(21) Appl. No.: 10/812,510

(22) Filed: Mar. 30, 2004

(51) Int. Cl. A61K 38/48

(52) U.S. Cl. 424/94.64

(57) ABSTRACT

The present invention provides a method for the treatment of acute ischemic stroke based on a specific rate and pattern of defibrinogenation and subsequent refibrogenation associated with optimized efficacy and significantly improved safety. The administration of a single dose of defibrinogenating agent to achieve an optimal rate of defirinogenation avoids prolonged hypofibrinogenenemia and concomitant adverse side effects such as intracranial hemorrhage. The defibrinogenating agent is administered as a single dose and time-weighted end of treatment fibrinogen levels of >70 mg/dL are achieved by simply stopping administration of the defibrinogenating agent.

### FIELD OF THE INVENTION

The present invention relates to a method for the treatment of stroke by the administration of a single dose of a defibrinogenating agent followed by normalization of fibrinogen levels without the further administration of a defibrinogenating agent. More particularly, the invention relates to a method for minimizing neurological damage associated with stroke and reducing the likelihood and occurrence of serious side effects associated with stroke, such as symptomatic intracranial hemorrhage and death.

### BACKGROUND OF THE INVENTION

Stroke is a type of cardiovascular disease that affects the arteries leading to and within the brain. Ischemic stroke is the third leading cause of death and the leading cause of disability in the U.S. Acute ischemic stroke is due to angiographically visible embolic or thrombotic occlusion in 70% to 80% of cases. Following occlusion, there is a time-dependent cascade of pathological events characterized by decreased energy production, over-stimulation of neuronal glutamate receptors, intraneuronal accumulation of sodium, chloride and calcium ions, mitochondrial injury, and finally cell death. Critical ischemia produces a core of dead brain tissue surrounded by hypoxic but potentially salvageable tissue. The aim of treatment is to prevent or slow the cascade of events, and to restore normal blood flow as soon as possible. There are several treatment options, depending on the circumstances.

Intravenous ancrod for treatment of acute ischemic stroke has been reported (Sherman et al.; JAMA 283(18):2395-2403. Ancrod (known commercially by the trade names, ARWIN®, ARVIN®, and VIPRINEX®), a highly glycosylated serine protease having an average molecular weight of about 38,000 Dalton and a carbohydrate content of about 38%, is a fibrinogen-splitting enzyme obtained from the venom of the Malayan pit viper (Calloselasma rhodostoma, previously known as Agkistrodon rhodostoma or Ancistrodon rhodostoma) and which has anticoagulant properties and the ability to dissolve blood clots (J. Biochem; 131: 799, 1973).

Normal coagulation of blood is effected by thrombin, which eliminates fibrinopeptides A and B from the fibrinogen molecule and thus leads to the formation of fibrin (EP-B-0 556 906), the main constituent of thrombi in addition to, for example, red blood corpuscles or platelets. In contrast to thrombin, ancrod cleaves only the arginine-glycine linkage in the α-(“A”) chain of the fibrinogen molecule, which liberates fibrinopeptides A, AP and AY (Cole et al., J. Vascular. Surgery, 17: 288-292 1993). The β-(B) chain of the fibrinogen molecule is not attacked by ancrod and is thus not liberated. The fragments (de-“A”-fibrin monomers) produced after the elimination of the fibrinopeptides by ancrod are eventually able to polymerize to thin filaments (FIG. 1). Lacking an effect on factor XIII (with cross-linking activity), on other coagulation factors, and on platelets (Table 1)¹¹⁻¹³ the resulting fibrin polymers are rapidly digested by plasmin and eliminated from the circulation via the reticuloendothelial system^{14,15}. Further cleavage of the de-“A”-fibrinogen molecule by thrombin to give natural fibrin no longer takes place because the resulting molecule is not a thrombin substrate.

TABLE 1
Comparison of Effects of Ancrod and Thrombin

| Action | Ancrod | Thrombin |
|---|---|---|
| Converts fibrinogen to fibrin polymer | + | + |
| Polymer end-to-end bonds | + | + |
| Polymer end-to-end and end-to-side | − | + |
| Fibrinopeptide A release | + | + |
| Fibrinopeptide B release | − | + |
| Degrades fibrinogen into fragments (in vitro) | − | + |
| Stimulates fibrinogen synthesis | − | + |
| Activates plasminogen (in vitro) | − | + |
| Induces plasminogen activation (in vivo) | + | + |
| Induces platelet aggregation | − | + |
| Induces platelet release | − | + |
| Decreases platelet count (in vivo) | − | + |
| Factor VIII-, factor XIII activation | − | + |
| Factor VIII inactivation | − | + |
| Decrease of factors II, V, VII, VIII, IX, X | − | + |
| and XIII | | |

Ancrod causes a dose-dependent decrease in the blood fibrinogen concentration. Therapeutically induced and controlled hypofibrinogenemia diminishes the plasma viscosity and tendency of erythrocytes to aggregate so far that the flow properties of the blood are crucially improved. This provides the condition for greater flow of blood through stenosed vessels. Ancrod is currently approved to treat, for example, chronic disturbances of peripheral arterial blood flow and heparin-induced thrombocytopenia, and is undergoing clinical phase III studies on acute ischemic stroke.

The decreases in plasma plasminogen and α₂-antiplasmin levels, which are parallel to the decline in plasma fibrinogen with ancrod treatment, indicate that the endogenous fibrinolytic system is strongly activated. Indeed, in a study in human volunteers, Prentice et al² demonstrated that ancrod treatment not only reduced plasminogen and α₂-antiplasmin levels but also produced a rise in fibrin(ogen) degradation products and fibrinopeptide Bβ15-42, clear indicators of plasmin-mediated fibrinolysis. Sozka et al¹⁹ similarly showed an indirect fibrinolytic action in an umbilical vein endothelial model.

Berliner et al²⁰ demonstrated that defibrinogenation by ancrod reduces the state of leukocyte adhesiveness and aggregation. Chang and Huang²¹ observed a dose-dependent prolongation of the time lapse for inducing platelet plug formation in the microcirculation. The results of both these studies can be explained by improved hemorrheology due to reduced blood viscosity after ancrod infusion. Current methods of treatment utilize administration of multiple doses of ancrod over extended periods of time to reduce initial fibrinogen levels and to maintain a state of hypofibrinogenenemia, which is sometimes associated with adverse side effects intracranial hemorrhage.

### SUMMARY OF THE INVENTION

The present invention provides an improved method for the treatment of acute ischemic stroke based on a specific rate and pattern of defibrinogenation and subsequent refibrogenation associated with optimized efficacy and significantly improved safety. The administration of a single dose of defibrinogenating agent within defined parameters achieves an optimal rate of defibrinogenation and avoids prolonged hypofibrinogenenemia and concomitant adverse side effects such as intracranial hemorrhage.

In one aspect, therefore, the invention relates to a method for treating stroke that employs a method of administration of a defibrinogenating agent that achieves a desired pattern of rapid defibrinogenation followed by normalization of blood fibrinogen levels.

In another aspect, the invention relates to a method for treating stroke, the method comprising administering a defibrinogenating agent to a patient in need of such treatment at a rate sufficient to achieve rapid initial defibrinogenation; ceasing administration of the defibrinogenating agent after about 30 minutes to 12 hours and subsequently, allowing normalization of fibrinogen levels to occur without further administration of the defibrinogenating agent.

In a related aspect, the invention relates to a method for treating stroke, the method comprising administering to a patient in need of such treatment a defibrinogenating agent in a single dose about 0.05-1.25 IU/kg/hr, more preferably about 0.1-0.2 IU/kg/hr and even more preferably, about 0.14-0.175 IU/kg/hr wherein normalization of fibrinogen levels is allowed to occur without further administration of the defibrinogenating agent.

In yet another related aspect, the invention relates to a method for treating stroke wherein the defibrinogenating agent is ancrod.

### BRIEF DESCRIPTION OF THE DRAWINGS

FIG. 1 is a schematic representation comparing the mechanisms of action of ancrod and thrombin on fibrinogen.

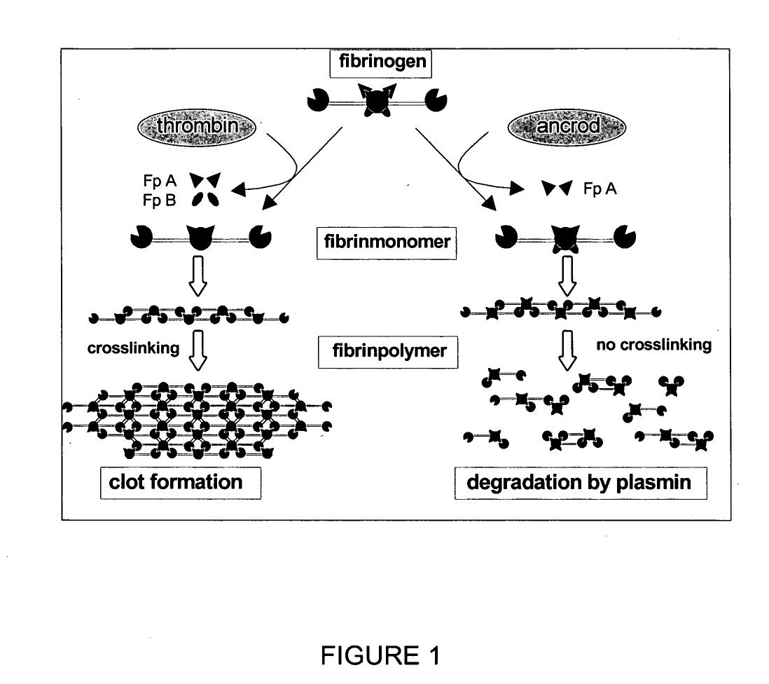

FIG. 2 depicts the effect on functional response, mortality and symptomatic intracranial hemorrhage (ICH) for a defibrinogenation rate of >30 mg/dL/hr (light gray bars) compared to a rate of <30 mg/dL/hr (dark gray bars.)

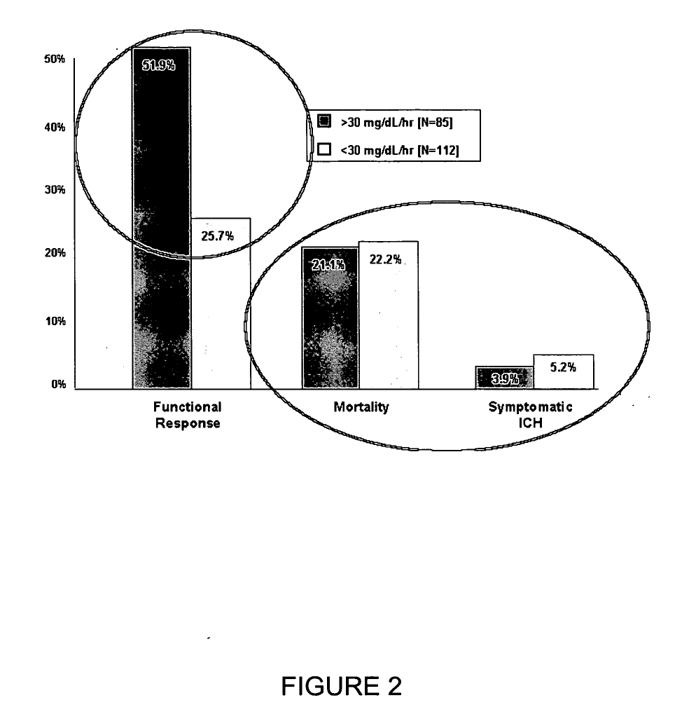

FIG. 3 depicts the difference between an average end-of treatment fibrinogen level of >60 mg/dL (dark gray bars) and <60 mg/dL (light gray bars) for functional response, mortality and symptomatic ICH.

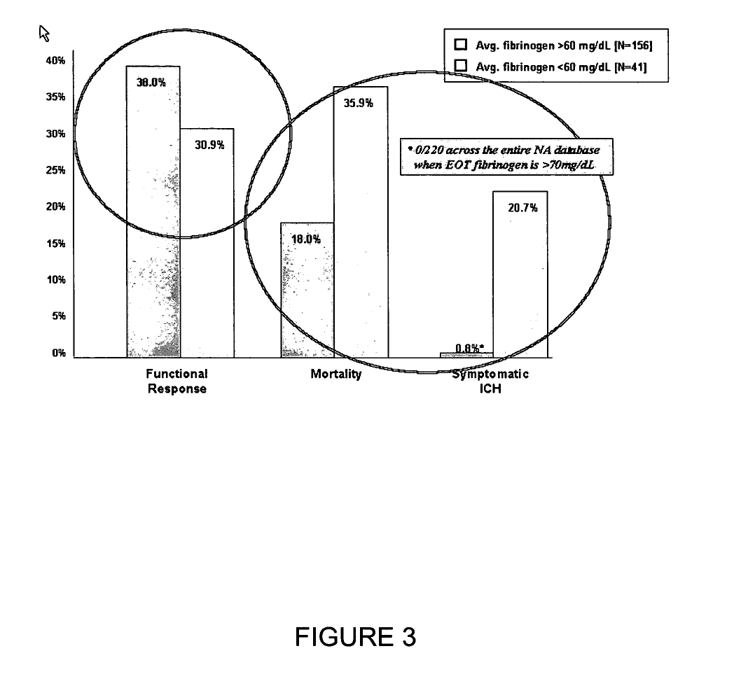

FIG. 4 is a representative graph of the effect on mean plasma fibrinogen concentrations of three different doses of ancrod administered as a single dose over a 12-hour time course. The broken line represents the effect (induction of hypofibrinogenemia) of the multiple-day dosing paradigm currently used in the art.

FIG. 5 is a graph representing the occurrence of intracranial hemorrhage when the target fibrinogen level was 40-69 mg/dL.

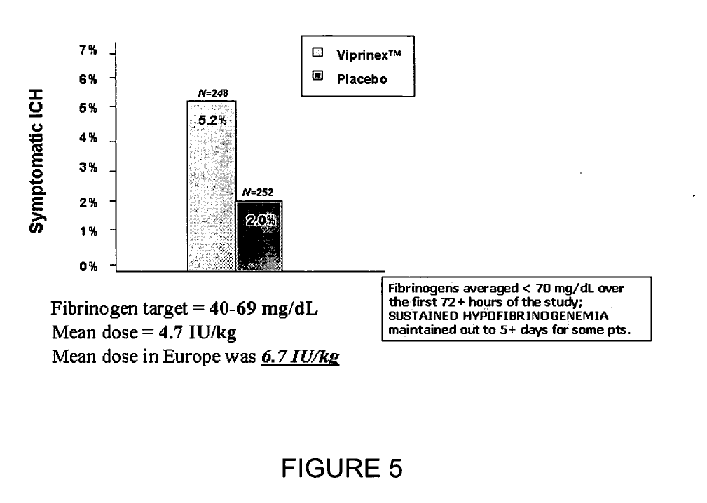

FIG. 6 is a graph representing the occurrence of intracranial hemorrhage when the target fibrinogen level was 70-100 mg/dL.

### DETAILED DESCRIPTION OF THE INVENTION

All patents, applications, publications and other references listed herein are hereby incorporated by reference in their entirety. In the description that follows, certain conventions will be followed as regards the usage of terminology.

The term “rapid initial defibrinogenation” refers to a rate of defibrinogenation, that is, an hourly decrease in fibrinogen, in excess of 20 mg/dL/hr, more preferably in excess of 25 mg/dL/hr and most preferably in excess of 30 mg/dL/hr.

The term “normalization” as used herein with respect to fibrinogen levels, refers to the natural return to baseline levels, without additional intervention. The term, as used, however, does not necessary imply that the rate of refibrinogenation is comparable to a normal physiological rate in an untreated subject.

In one embodiment, ancrod is used as the defribrinogenating agent for practicing the method of the present invention. As used herein, the term “ancrod” encompasses not only products prepared from the ancrod protease isolated from the Malayan pit viper venom, but also any products containing ancrod proteins obtained through recombinant technology. Methods for the preparation of ancrod from snake venom are well known, and include, but are in no way limited to, the methods taught in U.S. Pat. Nos. 6,200,791; 6,015,685; 3,743,722 and 3,879,369; Great Britain Patent documents 1,094,301; 1,177,506 and 1,293,793; and German patent documents 2,428,955 and 2,734,427. Methods for the preparation of ancrod products through genetic manipulation are taught, for example, in U.S. Pat. No. 5,759,541.

The present invention provides a method for the treatment of acute ischemic stroke based on a specific rate and pattern of defibrinogenation and subsequent refibrogenation that is associated with increased efficacy and safety. The administration of a single dose of defibrinogenating agent, usually by intravenous infusion, over a period of from about 15 minutes to 12 hours and more preferably from 30 minutes to 6 hours, achieves an optimal rate of defibrinogenation. Additionally, cessation of administration of defibrinogenating agent after the desired time avoids prolonged hypofibrinogenenemia and concomitant adverse side effects such as intracranial hemorrhage.

Therefore, the method of the present invention provides for a treatment of acute ischemic stroke wherein a defibrinogenating agent is administered as a single dose. At the end of the infusion period, time-weighted end of treatment fibrinogen levels of >50 mg/dL, more preferably >60 mg/dL, and most preferably >70 mg/dL, are achieved by simply stopping administration of the defibrinogenating agent.

In patients receiving ancrod as the defibrinogenating agent, blood viscosity is progressively reduced by 20-30% of the pretreatment levels. The diminished viscosity is directly attributable to the lowered fibrinogen levels and leads to an improvement in blood flow and microcirculation. Viscosity approaches pretreatment levels very slowly (approximately 10 days) after stopping ancrod.

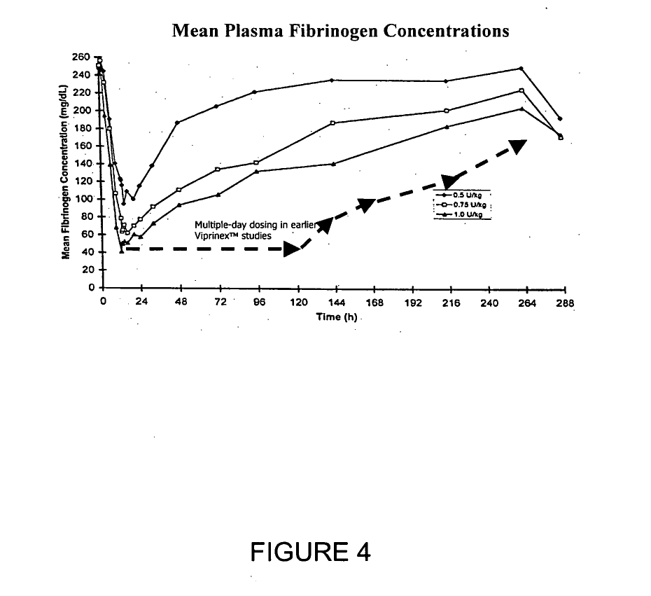

In four clinical pharmacological studies, 84 healthy adult volunteers received single doses of ancrod. Single infusions in different doses (0.125-1.0 IU/kg) administered over 3 to 24 hours produced a dose-related decrease in plasma fibrinogen with nadir levels occurring within 6 hours of stopping the infusion (FIG. 4). The gradual return of plasma fibrinogen to baseline after the end of the infusion was biphasic. Plasma fibrinogen returned to levels of 100 mg/dL within 72 hours and to baseline levels within 6-12 days depending on the 12-hour dose.

The relationship between the ancrod dose and defibrinogenating effect appeared to be best described as non-linear over the entire range of doses studied. The rate and extent of defibrinogenation varied according to the rate at which ancrod was administered. With equivalent doses, a faster infusion rate resulted in somewhat greater defibrinogenating activity. With an ancrod dose of 1.0 IU/kg administered over 12 hours a fibrinogen level of 20-70 mg/dL was achieved in 100% of the subjects studied. Similarly, a lower dose of 0.75 IU/kg infused over a shorter period of 3 hours yielded comparable fibrinogen levels in all subjects.

Efforts to model the fibrinogen profile based on data from the North American stroke program were performed by Knoll A G in Germany. At that time, the desired profile was to achieve initially rapid defibrinogenation (≧30 mg/dL/hr) but then to maintain hypofibrinogenemia in a target range of 40-69 mg/dL for five days of treatment. The resulting dosing paradigm called for an initial infusion rate of 0.167 IU/kg/hr administered for a limited time that was determined by dividing the pretreatment fibrinogen level in mg/dL by 100, truncating the result, and then stopping the infusion after that number of hours (e.g., for a pretreatment fibrinogen level of 375 mg/dL, one would infuse for 3 hrs, and for a pretreatment fibrinogen level of 415 mg/dL, one would infuse for 4 hrs). At 12 hours, the infusion was to resume at one-tenth the initial rate (i.e., 0.0167 IU/kg/hr) in order to maintain hypofibrinogenemia in the target range.

These observations lead us to conclude that a single, relatively-rapid intravenous administration of ancrod over a period of up to 6-12 hours (dose and duration determined by pretreatment fibrinogen level) will optimize efficacy and significantly improve safety, particularly with regard to diminishing the potential for symptomatic intracranial bleeding.

In addition to the late-stage data referenced above, preclinical studies in animal stroke models further support the effectiveness and safety of single-administration ancrod. Phase I human studies support the fact that single-administration dosing of ancrod across a range of doses and administered for various durations (30 minutes, 1-hour, 2 hours, 4-hours, 6-hours and 24-hours) will consistently yield the desired pattern of de-/re-fibrinogenation, and avoid prolonged hypofibrinogenemia. Specifically, rapid initial defibrinogenation can be achieved, and the optimally safe and effective end-of-treatment fibrinogen average can then be achieved by stopping the drug and allowing fibrinogen levels to recover naturally over a period of days.

The results of studies of ancrod in acute, ischemic stroke show beneficial effects. Ancrod given within 6 hours of stroke onset was shown to be effective in improving neurological function as measured with the Scandinavian Stroke Scale (SSS) in a double-blind, placebo-controlled trial in 132 patients⁶ (patient-weighted reanalysis noted subsequently⁴).

Efficacy, defined as survival for 90 days and functional independence (Barthel Index [BI] scores of 95-100 or at least as good as the prestroke BI), was also demonstrated in a subsequent double-blind, placebo-controlled study of 500 patients randomized to ancrod or placebo within 3 hours of stroke onset⁴.

In a Phase II multi-center, double-blind, randomized trial (The Ancrod in Stroke Investigators [1992] multicenter study⁶ with subsequent reanalysis¹), 132 patients were treated with IV ancrod vs. placebo within 6 hours of the onset of acute, ischemic stroke. Patients with evidence of bleeding on pretreatment CT scans were excluded, but there were no exclusions for patients with CT signs of evolving brain infarction. Patients were to have a pretreatment SSS score less than 40, excluding gait, meaning that patients with mild stroke were excluded. The initial infusion was 0.5 IU/kg over 6 hours (0.083 IU/kg/h), followed by daily 30-minute infusions to maintain fibrinogen at 70-100 mg/dL. The primary endpoint was an improvement in neurological deficits assessed by the SSS score^{22,23} at 3-months.

The primary endpoint, baseline-corrected 3-month SSS score, was higher (better) in ancrod (39) than in placebo-treated (35) patients, a statistically significant difference (p=0.044, patient-weighted analysis); the published non-significant p-value was based on a center-weighted analysis, where an otherwise positive result was diluted by a single center with confounding results. The median 3-month BI was better in ancrod (85) than placebo patients (65) (p=0.07); the categorical measure of success, defined as the attainment of a perfect (or pre-stroke) BI (i.e., complete functional recovery), was achieved by 45% of patients treated with ancrod and only 28% of patients receiving placebo (p<0.05)^{4,6}.

Ancrod patients attaining 6-hour fibrinogen levels at or below the median value of 130 mg/dL had a median BI at 3 months of 95 (a perfect score in this study) compared with a median of 75 for ancrod patients with fibrinogen levels above the median.

Deaths within 1 year were also lower in patients receiving ancrod (8/64) than those on placebo (14/68), with mean days to death being 43±45 (SD) for ancrod vs. 14±12 for placebo. One patient in the placebo group developed a symptomatic intracranial hemorrhage in the context of treatment, and two more placebo patients had traumatic subdural hematomas months later; no ancrod patient experienced a symptomatic intracranial hemorrhage. One center accounted for a disproportionate number of ancrod deaths. With this center excluded from the analysis, ancrod showed a benefit at p<0.01.

A Phase III North American trial⁴, a multi-center, parallel-group, sequential, double-blind, randomized, placebo-controlled study of the safety and efficacy of intravenous (IV) ancrod given within 3 hours after the onset of acute, ischemic stroke was conducted in 500 patients.

Patients were included from both sexes, who were at least 18 years of age, with acute or progressing ischemic (non-hemorrhagic) cerebral infarction with a neurological deficit that lasted longer than 30 minutes and occurred within 3 hours prior to study drug administration. Patients were to have a pretreatment SSS score less than 40, excluding gait, meaning that patients with mild stroke were excluded. The primary exclusion criterion based on blood pressure required that pre-treatment systolic pressure be ≦185 mmHg and pre-treatment diastolic pressure be ≦105 mmHg.

Ancrod (70 IU/mL) or placebo was diluted in a ratio of one ampule in each 250 mL normal saline. Defibrinogenation was induced with one of three initial infusion rates based on the pretreatment fibrinogen level (0.083 IU/kg/h, 0.125 IU/kg/h, and 0.167 IU/kg/h). The infusion was continued for 72 hours, followed by intermittent infusions administered at 96 (±6) and 120 (±6) hours after the start of blinded therapy. The dosing regimen permitted frequent regulation of infusion rates based on fibrinogen levels in order to achieve controlled defibrinogenation to levels of 40-69 mg/dL. Fibrinogen levels were provided only to an unblinded dosing supervisor at each site responsible for adjusting dosage. The unblinded dosing supervisor adjusted the rates as follows: for fibrinogen levels above the target, ancrod infusion rates were increased in 25-50% increments and for fibrinogen levels below the target, ancrod infusion rates were decreased by similar decrements or replaced with saline. Placebo (normal saline) was administered in accordance with schedules derived from previously-treated ancrod patients.

The primary efficacy variable (treatment success) was defined as survival for 90 days with a Barthel Index (BI) of 95-100 or at least as high as the prestroke BI. Secondary efficacy variables were the numerical BI (at 3 months), SSS score (at 3 days and 3 months), and CT infarct volume (at 7-10 days).

The principal analysis was carried out on the entire 500 patients according to the intent-to-treat (ITT) principle. The primary efficacy variable was analyzed using a logistic model with terms for age, baseline SSS score, pooled center and treatment. Secondary and tertiary efficacy parameters were analyzed both with a Wilcoxon Rank Sum test and with a general linear model approach using the same terms (including interaction) as for the primary efficacy parameter.

All tests were 2-sided and carried out at the 0.05 level except for the tests of interaction which were carried out at the 0.10 level. The final test of the primary efficacy variable was carried out at the 0.0472 level to account for the two interim analyses.

The mean age of patients in this study was 72.8 years; 51% were male, 89% were Caucasian, and 63.8% had a moderate-to-severe stroke as indicated by a Scandinavian Stroke Scale score of 0-29. The majority (85%) of patients began study drug within ≦3 hours of the onset of stroke. Eighty-two (82) patients were treated >3 hours after onset, and 12 of these 82 patients were treated >3.5 hours after onset (the latest at 4.8 hours post stroke onset). Most of these “late” patients were enrolled shortly after the 3-hour window with real-time permission from the sponsor; for a few, the later time-to-treat resulted from subsequently-derived information indicating an earlier time of symptom onset.

Ancrod was shown to be effective in improving functional outcome in patients with acute ischemic stroke ^{1,24}. Based on the prespecified primary efficacy endpoint, ancrod was superior to placebo (p=0.041), with the treatment-by-center term retained in the model, p=0.012. Age and pretreatment SSS scores are known to be predictors of outcome from stroke and, based on placebo rates of treatment success in this study, they were. Therefore, covariate-adjusted proportions of treatment success were calculated to provide a better estimate of the true treatment effect with age and pretreatment SSS scores as the covariates. The covariate-adjusted proportion of treatment successes was 42.2% in ancrod patients and 34.4% in placebo patients. No single site was disproportionately responsible for the favorable treatment effect.

Ancrod-treated patients had a greater improvement in neurological function than placebo-treated patients at 3 months, as indicated by a numerically higher adjusted mean SSS score (ancrod, 36.7 vs. placebo, 33.6; p=0.159). In addition, ancrod-treated patients had a greater degree of functional independence than placebo-treated patients at 3 months, as indicated by a numerically higher adjusted mean BI score (ancrod, 79.5 vs. placebo, 66.5; p=0.057). Furthermore, ancrod-treated patients had a numerically lower mean infarct volume than placebo-treated patients (ancrod, 31.9 cc vs. placebo, 42.0 cc; p=0.135). Results of subgroup analyses, including age category (<65, 65-74, 75-84, >85), pretreatment SSS category (<20, 20-29, 30-39), sex, and time-to-treatment (<2, 2-3, >3) were similar, with a higher proportion of ancrod patients achieving treatment success than placebo patients in all patient subsets characterized by age category, pretreatment SSS category, sex, early infarct signs on the pretreatment CT scan, and time-to-treatment.

Analysis of observed endpoints was also conducted in view of the similar mortality in the two treatment groups. This analysis resulted in a higher adjusted mean SSS in the ancrod (30.0) than placebo (27.8) groups (p=0.070); a higher adjusted mean 3-month SSS in the ancrod (40.8) than placebo (38.9) groups (p=0.029); and a higher 3-month BI in the ancrod (91.3) than placebo (82.9) groups (p=0.007).

In addition, more patients treated with ancrod achieved complete functional recovery (BI of 100 or at prestroke level, covariate-adjusted proportion: 36.1%) than placebo patients (28.4%; p=0.024). By contrast, fewer ancrod-treated patients were severely disabled at 3 months (BI: 0-40; covariate-adjusted proportion: 11.8%) than placebo patients (19.8%; p=0.011).

Ancrod benefit extended to those patients whose study drug treatment was delayed beyond 3 hours after stroke onset⁴. More ancrod patients treated within 3.5 hours of stroke onset achieved treatment success than placebo (p=0.029). This preservation of treatment effect beyond 3 hours is consistent with positive results in other 6-hour ancrod trials (A-20) and suggests that small errors in determining the time of stroke onset are not likely to detract from the beneficial effects seen with ancrod treatment.

A relationship between fibrinogen level and efficacy was observed. There was a strong association between treatment success and early, rapid defibrinogenation (as opposed to absolute fibrinogen levels), and fibrinogen levels at 6 hours <130 mg/dL (p=0.079) or at 9 hours <70 mg/dL (target fibrinogen; p=0.073), independent of the effects of age and pretreatment stroke severity.

Analyses of the combined North American experience revealed that the combination of early and rapid defibrinogenation, with subsequent avoidance of prolonged defibrinogenation (hypofibrinogenemia), consistently yields an optimally safe and efficacious outcome.

In a randomized, double-blind, placebo-controlled European study (European Stroke Treatment with Ancrod Trial, unpublished), 1222 patients had been enrolled within 6 hours of onset of acute ischemic stroke. The treatment phase with ancrod was 5 days consisting of a 72-hour period of continuous infusion and two intermittent infusions 96 and 120 hours after starting treatment, as in the North American trials. The infusion rates were adjusted according to the frequently monitored fibrinogen levels to achieve a target range of 40-69 mg/dL. The primary endpoint was the proportion of patients achieving functional success (as in STAT); secondary endpoints were improvement in the Barthel Index and SSS score, and determination of death rates and safety parameters at 3 months. Some entry criteria (such as blood pressure) differed from the North American program.

In one subset of patients, namely those who were administered ancrod at the most rapid infusion rate, efficacy was statistically significantly better than placebo. Importantly, that same group of patients that received ancrod at the most rapid infusion rate also had the lowest rate of ICH (4.4%).

A retrospective, multivariate review of ancrod data was performed and variables related to ancrod's dosing and effect on fibrinogen levels that influence both safety and efficacy were identified. Data from North American stroke trials revealed that the administration of ancrod within established parameters provides a specific pattern of defibrinogenation followed by refibrinogenation and that this pattern of de- followed by re-fibrinogenation maximizes efficacy while dramatically reducing the risk of intracranial bleeding, mortality and other undesirable side effects. Even after adjusting for the important covariates of age and pretreatment stroke severity, the initial rate of defibrinogenation positively correlated with efficacy, and the depth of prolonged hypofibrinogenemia predicted symptomatic intracranial hemorrhage. Since these two variables need not themselves be correlated, this analysis suggests that rapid initial defibrinogenation followed by termination of ancrod administration will yield optimal efficacy and safety. As can be seen in Table 2, ancrod patients with rapid initial defibrinogenation (≧30 mg/dL/hr) had a functional response rate above 50% with no increase in either mortality or symptomatic ICH. Moreover, patients whose time-weighted end-of-treatment (EOT) fibrinogen levels were above 60 mg/dL from 9 hours through the end of ancrod treatment had a rate of symptomatic ICH similar to placebo but did not sacrifice anything with respect to efficacy and when EOT fibrinogen levels averaged >70 mg/dl, there were no symptomatic ICH's.

TABLE 2
Fibrinogens and Clinical Outcome-STAT Study

| | | Proportions Adjusted for Age and | | |
|---|---|---|---|---|
| | | Pretreatment Stroke Severity as | | |
| | | Continuous Variables | | |
| | | Functional | | Symptomatic |
| Criterion | | Response | Mortality | ICH |
| Initial (3-hour) | Yes | 51.90% | 21.10% | 3.90% |
| defibrinogenation | (n = 79) | | | |
| rate | No | 25.70% | 22.20% | 5.20% |
| ≧30 mg/dL/hr? | (n = 118) | | | |
| 9-hour to EOT | Yes | 38.00% | 18.00% | 0.00% |
| fibrinogen level | (n = 155) | | | |
| ≧70 mg/dL? | No | 30.90% | 35.90% | 20.70% |
| | (n = 41) | | | |

Significantly, there were no symptomatic ICHs among 220 patients across the entire North American database whose maintenance fibrinogen levels were over 70 mg/dL. These effects were independent of the effects of age and pretreatment stroke severity.

While not wishing to be bound by theory, it appears that it is not the absolute fibrinogen level that is significant in this dosing paradigm; rather, it is the specific rate and pattern of defibrinogenation and refibrinogenation achieved with specific rates of defibrinogenatin agent administration. The rate and extent of defibrinogenation varies according to the rate at which the defibrinogenating agent, for example, ancrod is administered. With equivalent doses, a faster infusion rate results in somewhat greater defibrinogenating activity. A near-linear relationship was observed between treatment success and early, rapid defibrinogenation (as opposed to absolute fibrinogen levels and fibrinogen levels at 6 hours ≦130 mg/dL (p=0.079) or at 9 hours <70 mg/dL (target fibrinogen; p=0.073), independent of the effects of age and pretreatment stroke severity. Furthermore, the analyses revealed that the combination of rapid, initial defibrinogenation, with avoidance of prolonged hypofibrinogenemia, yields optimally safe and efficacious outcomes.

In Table 3, we calculated the nominal hourly defibrinogenation rate and divided this by 30. Consequently, ratios <1.00 reflect patients who did not defibrinogenate at or above 30 mg/dL/hr, and ratios ≧1.00 reflect patients who did. The one patient experiencing symptomatic ICH has a 1 in that column; all others have a zero. The values are sorted by the defibrinogenation rate, again emphasizing the point that it is the rate of infusion, and not the absolute fibrinogen value, that is linked to outcome.

TABLE 3
First Hour Infusion >0.14 UY/kg/hr

| | | | | | Hourly | Symp | |
|---|---|---|---|---|---|---|---|
| | BL | 3 Hr | BL-3 Hr | Nominal | Rate/30 | ICH | Efficacy |
| 17-334 | 385 | 333 | 52 | 17.3 | 0.58 | 0 | no |
| 1-355 | 269 | 206 | 63 | 21.0 | 0.70 | 0 | no |
| 1-382 | 380 | 307 | 73 | 24.3 | 0.81 | 0 | no |
| 52-323 | 452 | 369 | 83 | 27.7 | 0.92 | 0 | no |
| 48-327 | 390 | 303 | 87 | 29.0 | 0.97 | 0 | yes |
| 4-355 | 356 | 260 | 96 | 32.0 | 1.07 | 0 | no |
| 48-324 | 484 | 386 | 98 | 32.7 | 1.09 | 0 | no |
| 17-305 | 489 | 390 | 99 | 33.0 | 1.10 | 0 | yes |
| 52-307 | 455 | 330 | 125 | 41.7 | 1.39 | 0 | no |
| 50-302 | 587 | 450 | 137 | 45.7 | 1.52 | 1 | no |
| 8-302 | 364 | 225 | 139 | 46.3 | 1.54 | 0 | yes |
| 24-313 | 450 | 308 | 142 | 47.3 | 1.58 | 0 | no |
| 52-311 | 476 | 326 | 150 | 50.0 | 1.67 | 0 | yes |
| 48-310 | 478 | 296 | 182 | 60.7 | 2.02 | 0 | yes |
| 4-334 | 335 | 149 | 186 | 62.0 | 2.07 | 0 | no |
| 48-308 | 463 | 275 | 188 | 62.7 | 2.09 | 0 | no |
| 34-305 | 456 | 253 | 203 | 67.7 | 2.26 | 0 | yes |
| 48-311 | 463 | 260 | 203 | 67.7 | 2.26 | 0 | yes |
| 29-301 | 471 | 259 | 212 | 70.7 | 2.36 | 0 | yes |
| 1-302 | 375 | 157 | 218 | 72.7 | 2.42 | 0 | yes |
| 43-333 | 482 | 262 | 220 | 73.3 | 2.44 | 0 | no |
| 25-316 | 493 | 272 | 221 | 73.7 | 2.46 | 0 | no |
| 23-301 | 393 | 170 | 223 | 74.3 | 2.48 | 0 | no |
| 54-307 | 453 | 229 | 224 | 74.7 | 2.49 | 0 | yes |
| 15-311 | 500 | 268 | 232 | 77.3 | 2.58 | 0 | yes |
| 48-316 | 512 | 279 | 233 | 77.7 | 2.59 | 0 | yes |
| 29-306 | 590 | 346 | 244 | 81.3 | 2.71 | 0 | no |
| 6-309 | 516 | 256 | 260 | 86.7 | 2.89 | 0 | yes |
| 1-362 | 539 | 276 | 263 | 87.7 | 2.92 | 0 | no |
| 4-304 | 459 | 196 | 263 | 87.7 | 2.92 | 0 | yes |
| 15-307 | 555 | 280 | 275 | 91.7 | 3.06 | 0 | yes |
| 4-309 | 503 | 221 | 282 | 94.0 | 3.13 | 0 | no |
| 1-318 | 479 | 177 | 302 | 100.7 | 3.36 | 0 | no |
| 22-302 | 522 | 190 | 332 | 110.7 | 3.69 | 0 | yes |
| 15-305 | 1050 | 690 | 360 | 120.0 | 4.00 | 0 | no |
| | | | | | Efficacy | 16/35 | 46% |
| | | | | | Efficacy | 15/30 | 50% |
| | | | | | ICH | None | 0% |

As shown in Table 3, 31 of 36 patients (86.1%) who were infused at a rate of 0.167 IU/kg/hr achieved initial defibrinogenation ≧30 mg/dl/hr. Note that 15 of the 31 patients who achieved the target defibrinogenation had positive efficacy outcomes (an astonishing 48.4% with a small N) while only one of 5 patients (20%) outside the desired range was a treatment success (note however that the one success in the group was in fact at an hourly rate/30 of 0.97—very close to the desired cutoff point of 1.00). The occurrence of 1 ICH in this population is the same as that to be expected in a comparably sized placebo group.

It appears that the greatest efficacy stems from an hourly decrease in fibrinogen in excess of 20 mg/dL/hr, more preferably in excess of 25 mg/dL/hr, and even more preferably in excess of 30 mg/dL/hr. To that end, a rate of ancrod infusion of about 0.05-1.0 IU/kg/hr, more preferably about 0.1-0.5 IU/kg/hr and most preferred about 0.16-0.25 IU/kg/hr, consistently yields initial defibrinogenation at a rate of >30 mg/dl/hr, which is correlated to positive efficacy outcomes (i.e., a positive functional response rate of 50% alive with a Barthel Index score within 5 points of the prestroke score).

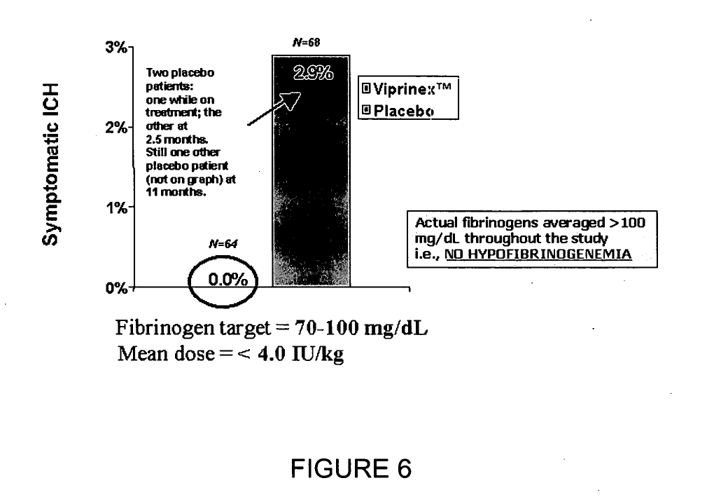

With respect to safety, a time-weighted average end-of-treatment fibrinogen level of 50 mg/dL or higher, and more preferably 60 mg/dL, and even more preferably 70 mg/dL, is associated with optimal safety outcomes. In a remarkable finding, there were no occurrences of symptomatic ICH in any of 220 ancrod patients across the North American experience who achieved a time-weighted average end-of-treatment fibrinogen level >70 mg/dL (FIG. 6).

It also appears that bleeding not related to bruising and, in particular, symptomatic intracranial hemorrhage (ICH) can best be avoided by maintaining average fibrinogen levels during the maintenance phase above 50 mg/dL. Therefore, while a desired rate of defibrinogenation of >30 mg/dl/hr associated with optimized efficacy can be consistently achieved with a specific rate of infusion over a period of from about 30 minutes to several hours, the desired time-weighted end of treatment fibrinogen level of >50 mg/dL can be consistently achieved by simply stopping the infusion.

### REFERENCES

1. Burkhart W, Smith G F H, Su J-L, Parikh I, LeVine H I. Amino acid sequence determination of ancrod, the thrombin-like à-fibrinogenase from the venom of Akistrodon-rhodostoma. FEBS Letters 1992; 297:297-301.
2. Prentice C R M, Hampton K K, Grant P J, Nelson S R, Nieuwenhuizen W, Gaffney P J. The fibrinolytic response to ancrod therapy: characterization of fibrinogen and fibrin degradation products. British Journal of Haematology 1993; 83:276-81.
3. Pollak V E, Glas-Greenwalt P, Olinger C P, Wadhwa N K, Myre S A. ancrod causes rapid thrombolysis in patients with acute stroke. American Journal of the Medical Sciences 1990; 299:319-25.
4. Sherman D G, Atkinson R P, Chippendale T et al. Intravenous ancrod for treatment of acute ischemic stroke: the STAT study: a randomized controlled trial. Journal of the American Medical Association 2000; 283:2395-2403.
5. Levy D E. Intravenous ancrod for the emergency treatment of acute, ischemic stroke: the North American experience. Trouillas P, Chair. 7th International Symposium on Thrombolysis and Acute Stroke Therapy. Lyon, 2002; 48.
6. The Ancrod Stroke Study Investigators. Ancrod for the treatment of acute ischemic brain infarction. Stroke 1994; 25:17455-1759.
7. Lewandowski C, Lotfipour S. Lessons learned from multicenter randomized clinical trials with intravenous thrombolysis for acute ischemic stroke. Journal of Stroke and Cerebrovascular Diseases 2002; 11:125-36.
8. Ewart M R, Hatton M W C, Basford J M, Dodgson K S. The proleolytic action of Arvin on human fibrinogen. Biochemical Journal 1970; 118:603-9.
9. Barlow G H, Holleman W H, Lorand L. The action of Arvin on fibrin stabilizing factor (Factor XIII). Research Communications in Chemical Pathology and Pharmacology 1970; 1:39-42.
10. Bell W R, Shapiro S S, Martinez J, Nossel H. The effects of ancrod, the coagulating enzyme from the venom of Malayan pit viper (A. rhodostoma) on prothrombin and figrinogen metabolism and fibrinopeptide A release in man. Journal of Laboratory and Clinical Medicine 1978; 91:592-604.
11. Kwaan H C, Barlow G H. The mechanism of action of Arvin and reptilase. Thrombosis Et Diathesis Haemorrhagica 1971; 47:361-9.
12. Bell W R, Bolton G, Pitney W R. The Effect of Arvin on blood coagulation factors. British Journal of Haematology 1968; 15:589-602.
13. Bell W R. Defibrinogenation with Arvin in thrombotic disorders. Platelets and Thrombosis. Baltimore: University Park Press, 1974: 273-98. (Sherry S, Scriabine A).
14. Silberman S, Bernik M B, Potter E V, Kwaan H C. Effects of ancrod (Arvin) in mice: Studies of plasma fibrinogen and fibrinolytic activity. British Journal of Haematology 1973; 24:101-13.
15. Lee L, Prose P H, Cohen M H. The role of the reticuloendothelial system in diffuse, low grade intravascular coagulation. Thrombosis Et Diathesis Haemorrhagica 1966; 20((suppl)):87.
16. Laux V, Elger B, Schwarz M, Hornberger W. Fibrinogen lowering with ancrod (Arvin(R)) in acute ischemic stroke: preclinical data confirm the efficient and safe rheological principle. Sixth World Congress for Microcirculation. Bologna: Monduzzi Editore, 1996: 297-300. (Messmer K, K bler W M).
17. Bell W R. Defibrinogenating enzymes. Hemostasis and Thrombosis: Basic Principles and Clinical Practice, 2nd Edition. Philadelphia: J.B. Lippincott Co., 1987: 886-900. (Colman R W, Hirsh J, Marder V J, Salzman E W).
18. Ehrly A M. Therapie chronischer peripherer arterieller Verschluβkrankheiten mit dem Schlangengift Enzym Arwin. Übersicht. Med. Welt. 1975; 26:446-55.
19. Soszka T, Kirschbaum N E, Stewart G J, Budzynski A Z. Effect of fibrinogen-clotting enzymes on secretion of plasminogen activators from cultured human endothelial cells. Fibrinolysis 1988; 2:49-57.
20. Berliner S, Fuchs J, Seligson U, et al. Possible role of fibrinogen in the aggregation of white blood cells. Thrombosis Haemostasis 1987; 87:749-52.
21. Chang M C, Huang T F. In vivo effect of a thrombin-like enzme on platelet plug formation induced in mesenteric microvessel of mice. Thrombosis Research 1994; 73:31-8.
22. Scandinavian Stroke Study Group. Multicenter trial of hemodilution in ischemic stroke—background and study protocol. Stroke 1985; 16:885-90.
23. Lindenstrom E, Boysen G, Christiansen L W, Hansen B R, Nielsen P W. Reliability of Scandinavian neurological stroke scale. Cerebrovascular Diseases 1991; 1:103-7.
24. Sherman D G, for the STAT Writers Group. Defibrinogenation with ancrod for the treatment of acute, ischemic stroke. (abst.). Stroke 1999; 30:234.
25. The National Institute of Neurological Disorders and Stroke rt-PA Stroke Study Group. Tissue plasminogen activator for acute ischemic stroke. New England Journal of Medicine 1995; 333:1581-7.
26. Hacke W, Kaste M, Fieschi C et al. Intravenous thrombolysis with recombinant tissue plasminogen activator for acute hemispheric stroke: the European Cooperative Acute Stroke Study (ECASS). Journal of the American Medical Association 1995; 274:1017-25.
27. Hacke W, Kaste M, Fieschi C et al. Randomised double-blind placebo-controlled trial of thrombolytic therapy with intravenous alteplase in acute ischaemic stroke (ECASS II). Lancet 1998; 352:1245-51.

1. A method for treating stroke said method comprising:
a) administering a defibrinogenating agent to a patient in need of such treatment at a rate sufficient to achieve rapid initial defribrinogenation;
b) ceasing administration of said defibrinogenating agent after about 15 min to 12 hours; and
b) allowing normalization of fibrinogen levels to occur without further administration of said defibrinogenating agent.

2. The method of claim 1, wherein initial defibrinogenation occurs at a rate of ≧20 mg/dL/hr.

3. The method of claim 1, wherein initial defibrinogenation occurs at a rate of ≧30 mg/dL/hr.

4. The method of claim 4, wherein the defibrinogenating agent is ancrod.

5. The method of claim 1 wherein the defibrinogenating agent is administered as a single dose of about 0.05-1.25 IU/kg/hr.

6. The method of claim 1 wherein the defibrinogenating agent is administered as a single dose of about 0.1-0.2 IU/kg/hr.

7. The method of claim 1 wherein the defibrinogenating agent is administered as a single dose at a rate of about 0.14-0.175 IU/kg/hr.

8. The method of claim 1 where the defibrinogenating agent is administered intravenously.

9. A method for treating stroke said method comprising:
a) administering a defibrinogenating agent to a patient in need of such treatment at a rate of about 0.10-1.5 IU/kg/hr;
b) ceasing administration of the defibrinogenating agent after about 15 min to 12 hours; and
c) allowing normalization of fibrinogen levels to occur without further administration of said defibrinogenating agent.

10. The method of claim 9 wherein said defibrinogenating agent is administered from about 30 minutes to 6 hours.

11. The method of claim 9 where the defibrinogenating agent is administered intravenously.
